(19) United States
(12) Patent Application Publication
(10) Pub. No.: US 2018/0021307 A1
(43) Pub. Date: Jan. 25, 2018

(54) Combination Treatment

(71) Applicant: UCB Biopharma SPRL, Brussels (BE)

(72) Inventors: Rafal Kaminski, Brussels (BE); Karine Leclercq, Brussels (BE); Frank Dressen, Brussels (BE)

(21) Appl. No.: 15/549,345

(22) PCT Filed: Feb. 19, 2016
(86) PCT No.: PCT/EP2016/053525

§ 371 Date: Aug. 7, 2017

(30) Foreign Application Priority Data
Feb. 20, 2015 (EP) 15155955.6

(51) Int. Cl. A61K 31/4015; A61K 31/165

(52) U.S. Cl. CPC A61K 31/4015; A61K 31/165; A61K 2300/00; A61K 9/2004

(57) ABSTRACT

The present invention relates and the combination of lacosamide and brivaracetam for the preparation of a fixed dose combination that is useful in the treatment of epilepsy, epileptogenesis, seizure disorders and convulsions.

Epilepsy refers to a clinical phenomenon rather than a single disease entity and describes a condition in which a person has recurrent seizures due to a chronic, underlying process.

Four subdivisions of epilepsy are recognized: grand mal epilepsy (with subgroups: generalized, focal, jacksonian), petit mal epilepsy, psychomotor or temporal lobe epilepsy (with subgroups: psychomotor proper or tonic with adversive or torsion movements or masticatory phenomenon, automatic with amnesia, or sensory with hallucinations or dream states) and autonomic or diencephalic epilepsy (with flushing, pallor, tachycardia, hypertension, perspiration or other visceral symptoms).

While epilepsy is one of the foremost examples of a seizure-related disorder, a wide variety of neurological and psychiatric symptoms and disorders may have, as their etiology, seizures or related seizure-like neurological phenomenon. In simple terms, a seizure or a related seizure-like neurological phenomenon is a single discrete clinical event caused by an excessive electrical discharge from a collection of neurons or a seizure susceptible group of neurons through a process termed “ictogenesis.” As such, ictogenic seizures may be merely the symptom of a disease. However, epilepsy and other analogous seizure-related disorders are dynamic and often progressive diseases, with a maturation process characterized by a complex and poorly understood sequence of pathological transformations.

The development and maturation of such changes is the process of “epileptogenesis,” whereby the larger collection of neurons that is the normal brain is altered and subsequently becomes capable of generating abnormal, spontaneous, sudden, recurrent, excessive electrical discharges, i.e., seizures. The maturation of the epileptogenic process results in the development of an “epileptogenic focus,” whereby the collections of abnormally discharging neurons or neurons susceptible to seizures form localized groups or “epileptogenic zones” interspersed throughout the cortical tissue. The epileptogenic zones are biochemically inter-connected such that an abnormal ictogenic discharge is able to cascade from zone to zone.

As epileptogenesis progresses, the involved areas of the nervous system become more excitable and it becomes easier for a seizure to be triggered, resulting in progressively debilitating symptoms of the seizure or seizure-related disorder.

While ictogenesis and epileptogenesis may have a common origin in certain biochemical phenomenon and common neuronal pathways in various diseases, the two processes are not identical. Ictogenesis is the initiation and propagation of a seizure in a discrete time and space, a rapid and definitive electrical/chemical event that occurs over a period of time ranging from seconds to minutes.

Comparatively, epileptogenesis is a gradual biochemical or neuronal restructuring process whereby the normal brain is transformed by ictogenic events into an epileptogenically focused brain, having neuronal circuitry that becomes sensitized and responsive to ictogenic events, making an individual increasingly susceptible to the recurrence of spontaneous, episodic, time-limited seizures, resulting in progressively debilitating symptoms of the seizure or seizure-related disorder and progressive non-responsiveness to treatment. The maturation of an “epileptogenic focus” is a slow biochemical and/or structural process that generally occur over months to years. Epileptogenesis is a Two Phase Process:

“Phase 1 epileptogenesis” is the initiation of the epileptogenic process prior to the first epileptic seizure or symptom of an analogous seizure-related disorder, and is often the result of some kind of injury or trauma to the brain, i.e., stroke, disease (e.g., infection such as meningitis), or trauma, such as an accidental blow to the head or a surgical procedure performed on the brain.

“Phase 2 epileptogenesis” refers to the process during which brain tissue that is already susceptible to epileptic seizures or seizure related phenomena of an analogous seizure-related disorder, becomes still more susceptible to seizures of increasing frequency and/or severity and/or becomes less responsive to treatment. While the processes involved in epileptogenesis have not been clearly identified, it is believed by many scientists that the up regulation of excitatory coupling between neurons, mediated by N-methyl-D-aspartate (NMDA) receptors, is involved. Other scientists implicate down regulation of inhibitory coupling between neurons, mediated by gamma-amino-butyric acid (GABA) receptors. Many other factors may be involved in this process relating to the presence, concentration or activity of NO (nitric oxide) or iron, calcium or zinc ions.

Although epileptic seizures are rarely fatal, large numbers of patients require medication to avoid the disruptive, and potentially dangerous consequences of seizures. In many cases, medication used to manage the epileptic seizures or symptoms of an analogous seizure-related disorder is required for extended periods of time, and in some cases, a patient must continue to take such prescription medication for life. Furthermore, such drugs are only effective for the management of symptoms and have side effects associated with chronic, prolonged usage.

Accepted drugs for the treatment of epilepsy are anticonvulsant agents or, more properly termed, anti-epileptic drugs (AEDs), wherein the term “anti-epileptic” is synonymous with “anti-seizure” or “anti-ictogenic”. These drugs therapeutically suppress seizures by blocking the initiation of a single ictogenic event. But those AEDs now clinically available, do not prevent the process of epileptogenesis. In treating seizures or related symptoms of analogous seizure-related disorders, that is for diseases and disorders with seizure-like neurological phenomenon that may apparently be related to seizures disorders, such as mood cycling in Bipolar Disorder, impulsive behavior in patients with Impulse Control Disorders or for seizures resulting from brain injury, some AEDs may also be therapeutically useful. However, those AEDs now approved are unable to prophylactically or therapeutically prevent the initial development or progressive maturation of epileptogenesis to an epileptogenic focus that also characterizes analogous seizure-related disorders.

A wide variety of AEDs are available for the management of epileptic seizures and include older agents such as phenytoin, valproate and carbamazepine, as well as newer agents such as felbamate, gabapentin, topiramate, levetiracetam and tiagabine.

Lacosamide (LCM, R-2-acetamido-N-benzyl-3-methoxypropionamide) is a new AED which was approved by numerous regulatory authorities since 2008 for the adjunctive treatment of partial-onset seizures and was furthermore approved for monotherapy of POS in the US in 2014. Lacosamide enhances the slow inactivation of voltage-gated sodium channels without affecting the fast inactivation of voltage-gated sodium channels. The anticonvulsant activity, prior to confirmation in large clinical studies, was initially shown in animal models of epilepsy, including maximal electroshock seizure [MES], the 6 Hz refractory seizure model, and sound-induced seizure in Frings mice (Bialer et al., 2001, 2002; Hovinga 2003). Further, LCM is active against refractory self-sustaining status epilepticus. In addition to the activity of the drug in electrically induced seizures, it is effective against cobalt-homocysteine- and lithium-pilocarpine-induced status epilepticus (Bialer et al., 2001, 2002).

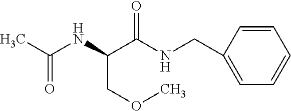 [SMILES: COC[C@@H](NC(C)=O)C(=O)NCc1ccccc1]

Lacosamide (Vimpat®) was approved in the following dosages: 50 mg, 100 mg, 150 mg and 200 mg in tablet form as well as 10 mg/ml as oral solution in the US and 15 mg/ml in the EU as well as in a strength of 200 mg/20 ml as iv solution. Typically, lacosamide is taken twice daily: 50 mg or 100 mg or 150 mg or 200 mg in the morning and 50 mg or 100 mg or 150 mg or 200 mg in the evening whereby the approved daily maintenance doses are 200 and 400 mg/day.

Brivaracetam (BRV, (2S)-2-((4R)-2-oxo-4-n-propyl-1-pyrrolidinyl) butanamide) is another new AED, currently awaiting regulatory approval for the treatment of epileptic seizures. The molecule is a modulator of the synaptic vesicle protein SV2A and was first disclosed in WO 01/62726.

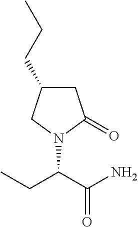

The following dosages are intended for approval: 25 mg, 50 mg and 100 mg in tablet form as well as 300 ml with 10 mg/ml in syrup form and 5 mL with 50 mg as iv solution.

Typically, brivaracetam is suggested to be administered as follows: twice daily 25 mg or 50 mg or 100 mg in the morning and 25 mg or 50 mg or 100 mg in the evening.

A persistent problem in seizure control arises with those patients who do not at all or only insufficiently respond to currently available treatments. Those patients are viewed as being refractory to treatment and represent a considerable challenge for the medical community. It is estimated that about 30% of epilepsy patients are to be classified as being refractory. Hence, there is a need to develop new medications that specifically target this population of patients.

In this logic of maximization of seizure control, in particular with a focus on essentially refractory patients, approximately 30% of epileptic patients are prescribed polytherapy regimens, i.e. administration of at least two AEDs. There is a clear need to develop a rational basis for AED polytherapy, i.e. to develop anticonvulsant compositions with improved effectiveness by improving efficacy or tolerability or reduce unwarranted side-effects. Effective AED combinations were empirically evaluated in patients with intractable seizures; however, such evaluations were often accompanied with deleterious adverse-effect reactions.

Further to polytherapy regimens, fixed-dose combinations (FDCs) are considered as an approach for maximizing seizure control. In fixed-dose combinations, multiple drugs (APIs) are combined into a single pill, which aims at helping to reduce the pill burden. FDCs are mainly prevalent in the field of anti-retrovirals, where they comprise different classes of anti-retrovirals or contain at least two drug molecules (API) of a single class. In the field of AEDs, no fixed-dose combination was approved by FDA or EMA so far.

FDCs to be used for the prevention, alleviation or/and treatment of epileptic seizures wherein the effect of this composition should display synergistic effect or a co-action compared to the effect of the individual APIs given alone. The present invention concerns fixed-dose combinations comprising two AEDs of a different class for the prevention, alleviation, minimization or/and treatment of epileptic seizures optionally together with a pharmaceutically acceptable carrier, diluent or/and adjuvant. The effect of this composition in the prevention, alleviation or/and treatment of epileptic seizures may be synergistic as compared to the effect of both AEDs given alone. FDCs may also provide patient value in as far as they provide a treatment approach for e.g. some difficult-to-treat forms of epilepsy, as well as on the minimization of side effects, including the benefit of adding two APIs with non-overlapping adverse event profiles, and of lower doses per drug.

A typical challenge for FDCs is the compatibility of both APIs from a galenical point of view. The main challenge when designing a single-combination therapy are the relative dissolution rates of the components (APIs) within the combination tablet, so that optimum drug concentrations are present in the bloodstream at the appropriate time, the potential impact of a respective component on the other components chemical stability, solubility, compactibility, tablet size.

The pharmacokinetics of some APIs depend critically on their formulation. Marked difference in the maximal plasma concentration between APIs to form a FDC. Factors like micronised and non-micronised particle preparations will have to be taken into account.

A modified release formulation is a formulation showing a release of the active substance(s), which is deliberately modified by a special formulation design or manufacturing method. This modified release can be typically obtained by delaying the time of release of one or both of the components. Typically, a modified release formulation may allow the release of therapeutically effective amounts of the active ingredients from the formulation over an extended period of time, e.g. for more than 6 hours, 9 hours, 12 hours, 18 hours, 24 hours or even more, in order to allow for a once daily or once every two days administration of the modified release formulation, whereas, if the drug release were not delayed by the formulation, a twice daily or more frequent administration of this immediate release formulation would be necessary. Modified release is meant to encompass both a different continuous release over time of the two components or a delayed release where one of the components is released only after a lag time. Such a modified release form may be produced by applying release-modifying coatings, e.g. a diffusion coating, to the drug substance(s) or to a core containing the drug substance(s), or by creating a release-modifying matrix embedding the drug substance(s).

In a nutshell, there is a need to provide an effective treatment for epilepsy, the prevention of epilepsy, epileptogenesis and related disorders, and preferably treatment which does not have less or no associated side-effects.

### SUMMARY OF THE INVENTION

The present invention relates to the combination of lacosamide and brivaracetam for the preparation of a fixed dose combination that is useful in the treatment of epilepsy, epileptogenesis, seizure disorders and convulsions.

### FIGURES

FIG. 1: Isobologram showing interactions between Brivaracetam (BRV) and Lacosamide (LCM) for three fixed-ratio combinations in the 6 Hz induced seizure model in mice. Median effective dose (ED50) values for Lacosamide and Brivaracetam are placed on the X- and Y-axes, respectively. The straight line connecting these both ED50 values represents the theoretic line of additivity for a continuum of different fixed-dose ratios. The solid points depict the experimentally derived ED50mix values (with 95% confidence limits as the error bars) for total dose expressed as the proportion of Lacosamide and Brivaracetam that produce a 50% effect.

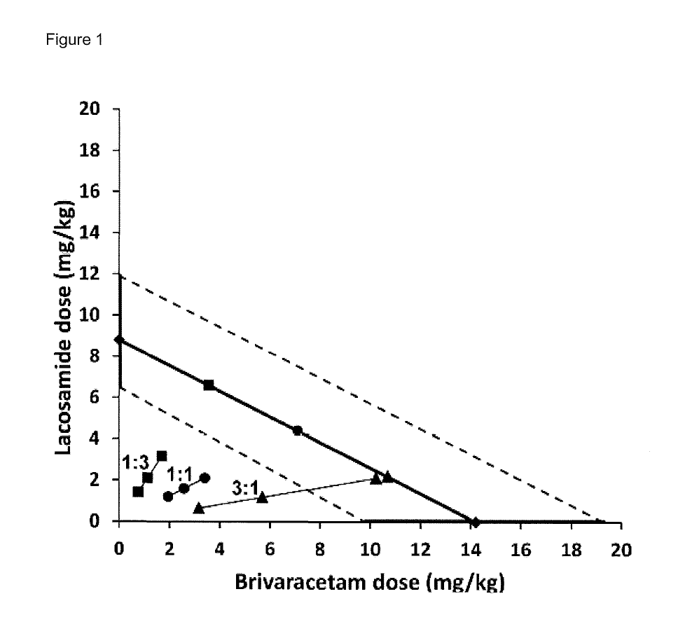

FIG. 2: Comparisons of efficacy between Brivaracetam (BRV), Lacosamide (LCM) and their fixed dose combinations (COMBO) in the rat amygdala kindling model. Top panels show effects on seizure severity score according to the Racine's scale, while bottom panels show fraction (%) of animals protected against secondarily generalized seizures in this model. From left to right: effects of Lacosamide and Brivaracetam alone (both at 20 mg/kg) or their combination; Lacosamide and Brivaracetam alone (both at 40 mg/kg) or their combination; BRV (20 mg/kg) and LCM (60 mg/kg) or their combination.

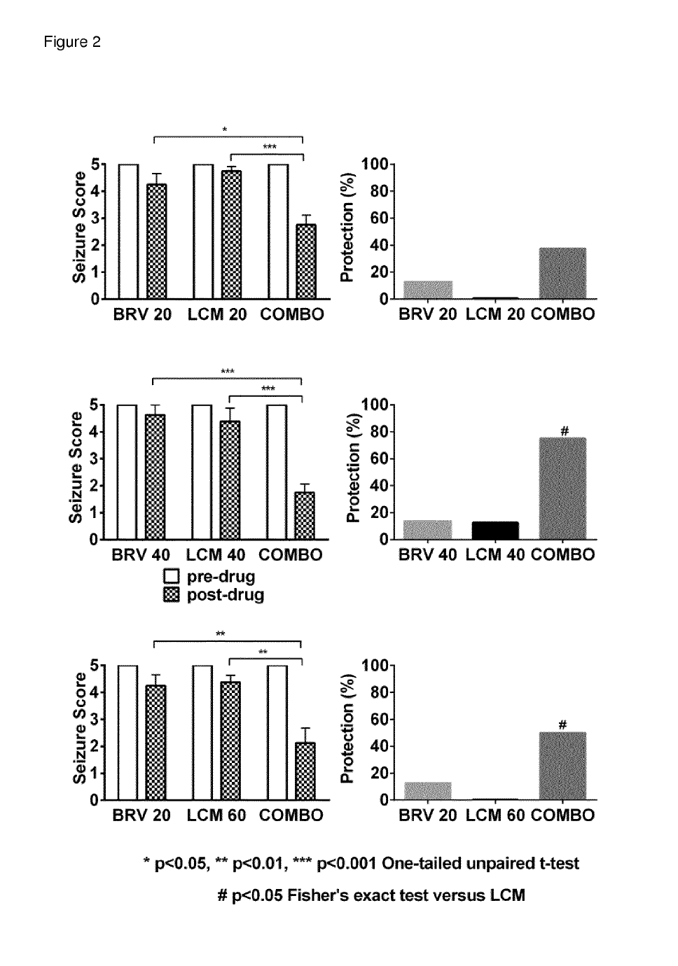

### DETAILED DESCRIPTION OF THE INVENTION

The present invention relates to a pharmaceutical oral fixed-dose combination—i.e. a single dosage form—comprising the combination of brivaracetam and lacosamide, as well as pharmaceutically acceptable excipients. In a first embodiment, all types of ratios and dosages of brivaracetam and lacosamide are comprised by the invention.

In a further embodiment the fixed-dose combination contains a surplus of lacosamide versus brivaracetam on a weight by weight basis; in another embodiment the fixed-dose combination contain a surplus of brivaracetam versus lacosamide on a weight by weight basis.

In a specific embodiment, a pharmaceutical oral fixed-dose combination in a single dosage form (e.g. a tablet or a capsule containing pellets) would comprise:
a) 5-150 mg brivaracetam
b) 5-250 mg lacosamide, and
c) pharmaceutically acceptable excipients.

In a specific embodiment, a pharmaceutical oral fixed-dose combination in a single dosage form (e.g. a tablet or a capsule containing pellets) would comprise:
a) 25-100 mg brivaracetam
b) 100-200 mg lacosamide, and
c) pharmaceutically acceptable excipients.

In a specific embodiment, a pharmaceutical oral fixed-dose combination in a single dosage form (e.g. a tablet or a capsule containing pellets) would comprise:
a) 50-80 mg brivaracetam
b) 120-150 mg lacosamide, and
c) pharmaceutically acceptable excipients.

In a specific embodiment, a pharmaceutical oral fixed-dose combination in a single dosage form (e.g. a tablet or a capsule containing pellets) would comprise:
a) 80-150 mg brivaracetam
b) 80-150 mg lacosamide, and
c) pharmaceutically acceptable excipients.

The above dosage forms may be administered to a patient, in need of, once or twice a day.

In another specific embodiment the pharmaceutical oral fixed-dose combination according to the present invention comprises a therapeutically ineffective amount of brivaracetam and a therapeutically effective amount of lacosamide in a single dosage, as well as pharmaceutically acceptable excipients. In a specific embodiment, the ineffective amount of brivaracetam would be below a dosage of 25 or 20 or 15 or 10 or 5 mg in a single dosage. Furthermore, in a specific embodiment the effective amount of lacosamide would be 50 mg, 100 mg, 150 mg, 200 mg or 250 mg in a single dosage, more preferably anywhere between 100 mg to 150 mg in a single dosage.

In a further specific embodiment the pharmaceutical oral fixed-dose combination according to the present invention comprises a therapeutically ineffective amount of lacosamide and a therapeutically effective amount of brivaracetam in a single dosage, as well as pharmaceutically acceptable excipients. In a specific embodiment, the ineffective amount of lacosamide in a single dosage would be below 20 mg. Furthermore, in a specific embodiment, the effective amount of brivaracetam in a single dosage would be 25 mg, 50 mg and 100 mg, more preferably about 100 mg.

In a further specific embodiment, the pharmaceutical oral fixed-dose combination according to present invention comprises a therapeutically ineffective amount of lacosamide and a therapeutically ineffective amount of brivaracetam in a single dosage, as well as pharmaceutically acceptable excipients. In a specific embodiment the ineffective amount of lacosamide in a single dosage would be below a dosage of 20 mg. Furthermore, in a specific embodiment the effective amount of brivaracetam in a single dosage would be below 25 or 20 or 15 or 10 or 5 mg.

In general, however, the total daily dose for brivaracetam in combination with lacosamide, for the conditions described herein, is about 10 mg or less up to about 200 mg of brivaracetam in combination with about 50 mg to about 250 mg or more lacosamide; preferably from about 15 or 20 mg to about 65, 70, 75, 80, 85, 90, 95, 100, 110, 120, 130, 140, 150, 160, 170, 180, 190 or 200 mg of brivaracetam in combination with from about 50 mg to about 55, 60, 65, 70, 75, 80, 85, 90, 95, 100, 110, 120, 130, 140 or 150 mg lacosamide; more preferably from about 25, 30, 35, or 40 mg to about 55 or 60 mg brivaracetam in combination with from about 100, 110, 120, 130, 140, 150 mg lacosamide.

A further aspect of the present invention consists in a treatment regimen wherein a single dosage form, comprising the combination of brivaracetam and lacosamide, as well as pharmaceutically acceptable excipients, is administered to a patient in need of. Said regimen aims at delivering a daily dose of said fixed dose combination of brivaracetam and lacosamide.

The term “daily dose” means the overall amount of brivaracetam in combination with lacosamide in the form of one or multiple single dosage forms that are given to a patient within a period of 24 hours.

A specific aspect of the present invention consists in fixed dose combinations of brivaracetam and lacosamide which are administered to a patient in such a way that the below daily doses are achieved:

20 mg brivaracetam and 20 mg lacosamide; 20 mg brivaracetam and 30 mg lacosamide; 20 mg brivaracetam and 40 mg lacosamide; 20 mg brivaracetam and 50 mg lacosamide; 20 mg brivaracetam and 60 mg lacosamide; 20 mg brivaracetam and 70 mg lacosamide; 20 mg brivaracetam and 80 mg lacosamide; 20 mg brivaracetam and 90 mg lacosamide; 20 mg brivaracetam and 100 mg lacosamide; 20 mg brivaracetam and 110 mg lacosamide; 20 mg brivaracetam and 120 mg lacosamide; 20 mg brivaracetam and 130 mg lacosamide; 20 mg brivaracetam and 140 mg lacosamide; 20 mg brivaracetam and 150 mg lacosamide. 20 mg brivaracetam and 160 mg lacosamide; 20 mg brivaracetam and 170 mg lacosamide; 20 mg brivaracetam and 180 mg lacosamide; 20 mg brivaracetam and 190 mg lacosamide; 20 mg brivaracetam and 200 mg lacosamide; 20 mg brivaracetam and 210 mg lacosamide; 20 mg brivaracetam and 220 mg lacosamide; 20 mg brivaracetam and 230 mg lacosamide; 20 mg brivaracetam and 240 mg lacosamide; 20 mg brivaracetam and 250 mg lacosamide.

30 mg brivaracetam and 20 mg lacosamide; 30 mg brivaracetam and 30 mg lacosamide; 30 mg brivaracetam and 40 mg lacosamide; 30 mg brivaracetam and 50 mg lacosamide; 30 mg brivaracetam and 60 mg lacosamide; 30 mg brivaracetam and 70 mg lacosamide; 30 mg brivaracetam and 80 mg lacosamide; 30 mg brivaracetam and 90 mg lacosamide; 30 mg brivaracetam and 100 mg lacosamide; 30 mg brivaracetam and 110 mg lacosamide; 30 mg brivaracetam and 130 mg lacosamide; 30 mg brivaracetam and 130 mg lacosamide; 30 mg brivaracetam and 140 mg lacosamide; 30 mg brivaracetam and 150 mg lacosamide; 30 mg brivaracetam and 160 mg lacosamide; 30 mg brivaracetam and 170 mg lacosamide; 30 mg brivaracetam and 180 mg lacosamide; 30 mg brivaracetam and 190 mg lacosamide; 30 mg brivaracetam and 200 mg lacosamide; 30 mg brivaracetam and 210 mg lacosamide; 30 mg brivaracetam and 220 mg lacosamide; 30 mg brivaracetam and 230 mg lacosamide; 30 mg brivaracetam and 240 mg lacosamide; 30 mg brivaracetam and 250 mg lacosamide.

40 mg brivaracetam and 20 mg lacosamide; 40 mg brivaracetam and 30 mg lacosamide; 40 mg brivaracetam and 40 mg lacosamide; 40 mg brivaracetam and 50 mg lacosamide; 40 mg brivaracetam and 60 mg lacosamide; 40 mg brivaracetam and 70 mg lacosamide; 40 mg brivaracetam and 80 mg lacosamide; 40 mg brivaracetam and 90 mg lacosamide; 40 mg brivaracetam and 100 mg lacosamide; 40 mg brivaracetam and 110 mg lacosamide; 40 mg brivaracetam and 140 mg lacosamide; 40 mg brivaracetam and 130 mg lacosamide; 40 mg brivaracetam and 140 mg lacosamide; 40 mg brivaracetam and 150 mg lacosamide; 40 mg brivaracetam and 160 mg lacosamide; 40 mg brivaracetam and 170 mg lacosamide; 40 mg brivaracetam and 180 mg lacosamide; 40 mg brivaracetam and 190 mg lacosamide; 40 mg brivaracetam and 200 mg lacosamide; 40 mg brivaracetam and 210 mg lacosamide; 40 mg brivaracetam and 220 mg lacosamide; 40 mg brivaracetam and 230 mg lacosamide; 40 mg brivaracetam and 240 mg lacosamide; 40 mg brivaracetam and 250 mg lacosamide.

50 mg brivaracetam and 20 mg lacosamide; 50 mg brivaracetam and 30 mg lacosamide; 50 mg brivaracetam and 40 mg lacosamide; 50 mg brivaracetam and 50 mg lacosamide; 50 mg brivaracetam and 50 mg lacosamide; 50 mg brivaracetam and 70 mg lacosamide; 50 mg brivaracetam and 80 mg lacosamide; 50 mg brivaracetam and 90 mg lacosamide; 50 mg brivaracetam and 100 mg lacosamide; 50 mg brivaracetam and 110 mg lacosamide; 50 mg brivaracetam and 150 mg lacosamide; 50 mg brivaracetam and 130 mg lacosamide; 50 mg brivaracetam and 140 mg lacosamide; 50 mg brivaracetam and 150 mg lacosamide; 50 mg brivaracetam and 160 mg lacosamide; 50 mg brivaracetam and 170 mg lacosamide; 50 mg brivaracetam and 180 mg lacosamide; 50 mg brivaracetam and 190 mg lacosamide; 50 mg brivaracetam and 200 mg lacosamide; 50 mg brivaracetam and 210 mg lacosamide; 50 mg brivaracetam and 220 mg lacosamide; 50 mg brivaracetam and 230 mg lacosamide; 50 mg brivaracetam and 240 mg lacosamide; 50 mg brivaracetam and 250 mg lacosamide.

60 mg brivaracetam and 20 mg lacosamide; 60 mg brivaracetam and 30 mg lacosamide; 60 mg brivaracetam and 40 mg lacosamide; 60 mg brivaracetam and 50 mg lacosamide; or 60 mg brivaracetam and 60 mg lacosamide; 60 mg brivaracetam and 70 mg lacosamide; 60 mg brivaracetam and 80 mg lacosamide; 60 mg brivaracetam and 90 mg lacosamide; 60 mg brivaracetam and 100 mg lacosamide; 60 mg brivaracetam and 110 mg lacosamide; 60 mg brivaracetam and 160 mg lacosamide; 60 mg brivaracetam and 130 mg lacosamide; 60 mg brivaracetam and 140 mg lacosamide; 60 mg brivaracetam and 150 mg lacosamide; 60 mg brivaracetam and 160 mg lacosamide; 60 mg brivaracetam and 170 mg lacosamide; 60 mg brivaracetam and 180 mg lacosamide; 60 mg brivaracetam and 190 mg lacosamide; 60 mg brivaracetam and 200 mg lacosamide; 60 mg brivaracetam and 210 mg lacosamide; 60 mg brivaracetam and 220 mg lacosamide; 60 mg brivaracetam and 230 mg lacosamide; 60 mg brivaracetam and 240 mg lacosamide; 60 mg brivaracetam and 250 mg lacosamide.

70 mg brivaracetam and 20 mg lacosamide; 70 mg brivaracetam and 30 mg lacosamide; 70 mg brivaracetam and 40 mg lacosamide; 70 mg brivaracetam and 50 mg lacosamide; or 70 mg brivaracetam and 60 mg lacosamide; 70 mg brivaracetam and 70 mg lacosamide; 70 mg brivaracetam and 80 mg lacosamide; 70 mg brivaracetam and 90 mg lacosamide; 70 mg brivaracetam and 100 mg lacosamide; 70 mg brivaracetam and 110 mg lacosamide; 70 mg brivaracetam and 160 mg lacosamide; 70 mg brivaracetam and 130 mg lacosamide; 70 mg brivaracetam and 140 mg lacosamide; 70 mg brivaracetam and 150 mg lacosamide; 70 mg brivaracetam and 160 mg lacosamide; 70 mg brivaracetam and 170 mg lacosamide; 70 mg brivaracetam and 180 mg lacosamide; 70 mg brivaracetam and 190 mg lacosamide; 70 mg brivaracetam and 200 mg lacosamide; 70 mg brivaracetam and 210 mg lacosamide; 70 mg brivaracetam and 220 mg lacosamide; 70 mg brivaracetam and 230 mg lacosamide; 70 mg brivaracetam and 240 mg lacosamide; 70 mg brivaracetam and 250 mg lacosamide.

80 mg brivaracetam and 20 mg lacosamide; 80 mg brivaracetam and 30 mg lacosamide; 80 mg brivaracetam and 40 mg lacosamide; 80 mg brivaracetam and 50 mg lacosamide; or 80 mg brivaracetam and 60 mg lacosamide; 80 mg brivaracetam and 70 mg lacosamide; 80 mg brivaracetam and 80 mg lacosamide; 80 mg brivaracetam and 90 mg lacosamide; 80 mg brivaracetam and 100 mg lacosamide; 80 mg brivaracetam and 110 mg lacosamide; 80 mg brivaracetam and 160 mg lacosamide; 80 mg brivaracetam and 130 mg lacosamide; 80 mg brivaracetam and 140 mg lacosamide; 80 mg brivaracetam and 150 mg lacosamide; 80 mg brivaracetam and 160 mg lacosamide; 80 mg brivaracetam and 170 mg lacosamide; 80 mg brivaracetam and 180 mg lacosamide; 80 mg brivaracetam and 190 mg lacosamide; 80 mg brivaracetam and 200 mg lacosamide; 80 mg brivaracetam and 210 mg lacosamide; 80 mg brivaracetam and 220 mg lacosamide; 80 mg brivaracetam and 230 mg lacosamide; 80 mg brivaracetam and 240 mg lacosamide; 80 mg brivaracetam and 250 mg lacosamide.

90 mg brivaracetam and 20 mg lacosamide; 90 mg brivaracetam and 30 mg lacosamide; 90 mg brivaracetam and 40 mg lacosamide; 90 mg brivaracetam and 50 mg lacosamide; or 90 mg brivaracetam and 60 mg lacosamide; 90 mg brivaracetam and 70 mg lacosamide; 90 mg brivaracetam and 80 mg lacosamide; 90 mg brivaracetam and 90 mg lacosamide; 90 mg brivaracetam and 100 mg lacosamide; 90 mg brivaracetam and 110 mg lacosamide; 90 mg brivaracetam and 160 mg lacosamide; 90 mg brivaracetam and 130 mg lacosamide; 90 mg brivaracetam and 140 mg lacosamide; 90 mg brivaracetam and 150 mg lacosamide; 90 mg brivaracetam and 160 mg lacosamide; 90 mg brivaracetam and 170 mg lacosamide; 90 mg brivaracetam and 180 mg lacosamide; 90 mg brivaracetam and 190 mg lacosamide; 90 mg brivaracetam and 200 mg lacosamide; 90 mg brivaracetam and 210 mg lacosamide; 90 mg brivaracetam and 220 mg lacosamide; 90 mg brivaracetam and 230 mg lacosamide; 90 mg brivaracetam and 240 mg lacosamide; 90 mg brivaracetam and 250 mg lacosamide.

100 mg brivaracetam and 20 mg lacosamide; 100 mg brivaracetam and 30 mg lacosamide; 100 mg brivaracetam and 40 mg lacosamide; 100 mg brivaracetam and 50 mg lacosamide; or 100 mg brivaracetam and 60 mg lacosamide; 100 mg brivaracetam and 70 mg lacosamide; 100 mg brivaracetam and 80 mg lacosamide; 100 mg brivaracetam and 90 mg lacosamide; 100 mg brivaracetam and 100 mg lacosamide; 100 mg brivaracetam and 110 mg lacosamide; 100 mg brivaracetam and 160 mg lacosamide; 100 mg brivaracetam and 130 mg lacosamide; 100 mg brivaracetam and 140 mg lacosamide; 100 mg brivaracetam and 150 mg lacosamide; 100 mg brivaracetam and 160 mg lacosamide; 100 mg brivaracetam and 170 mg lacosamide; 100 mg brivaracetam and 180 mg lacosamide; 100 mg brivaracetam and 190 mg lacosamide; 100 mg brivaracetam and 200 mg lacosamide; 100 mg brivaracetam and 210 mg lacosamide; 100 mg brivaracetam and 220 mg lacosamide; 100 mg brivaracetam and 230 mg lacosamide; 100 mg brivaracetam and 240 mg lacosamide; 100 mg brivaracetam and 250 mg lacosamide.

The above daily doses may be administered by a single dose per day or divided doses (two, three, four or more doses per day). A preferred embodiment would be to administer the fixed dose combination according and the present invention twice a day.

Preferably, a daily dose and achieve a significant anti-convulsive effect in human patients is about 20 mg and about 150 mg brivaracetam in combination with about 20 mg and about 200 mg lacosamide, in single or divided doses. Particularly preferred daily dose in epilepsy is about 20, 21, 22, 23, 24, 25, 26, 27, 28, 29, or 30 mg brivaracetam in combination with about 50, 60, 70, 80, 90, 100, 110, 120, 130, 140, or 150 mg lacosamide; about 30, 31, 32, 33, 34, 35, 36, 37, 38, 39, or 40 mg brivaracetam in combination with about 50, 60, 70, 80, 90, 100, 110, 120, 130, 140, or 150 mg lacosamide; about 40, 41, 42, 43, 44, 45, 46, 47, 48, 49, or 50 mg brivaracetam in combination with about 50, 60, 70, 80, 90, 100, 110, 120, 130, 140, or 150 mg lacosamide; or about 50, 51, 52, 53, 54, 55, 56, 57, 58, 59, or 60 mg brivaracetam in combination with about 50, 60, 70, 80, 90, 100, 110, 120, 130, 140, or 150 mg lacosamide; in single or divided doses.

The fixed dose combinations of this invention are preferably in tablet form.

In one embodiment the fixed dose combination of the present invention consists of a tablet comprising 30-50 mg of brivaracetam and 120-200 mg lacosamide or 50-80 mg of brivaracetam and 100-150 mg of lacosamide or 80 mg of brivaracetam and 150 mg lacosamide to be administered twice a day.

The term “synergistic effect on the prevention, alleviation or/and treatment of epileptic seizures” refers and an effect of the pharmaceutical composition according and the invention on the prevention, alleviation or/and treatment of epileptic seizures that is more than additive as compared and the effect of lacosamide and brivaracetam given alone.

Said synergy may furthermore entail the reduction of side effects as a consequence of the lower amounts of brivaracetam and lacosamide that are required to achieve essentially the same pharmacological effect which is the alleviation or/and treatment of epileptic seizures. Due to different mechanisms of action by brivaracetam and lacosamide, a further beneficial co-action may be due to non-overlapping side effect profiles.

The term “therapeutically ineffective amount of BRV” or the term “therapeutically ineffective amount of LCM” means that at the given dosage of BRV or LCM, there is no anti-convulsive effect in 50% of the population that takes it. Said term is analogue to “median effective dose” (ED50) that is the dose that produces a quantal effect (all or nothing; median referring to the 50% population base). It is also sometimes abbreviated as the ED50, meaning “effective dose, for 50% of people receiving the drug”. The ED50 is commonly used as a measure of the reasonable expectancy of a drug effect, but does not necessarily represent the dose that a clinician might use.

The skilled person may determine the ED50 values by methods known in the art. It is preferred that the ED50 values are determined by preclinical or/and clinical trials. Published ED50 values may also be used. ED50 values are published for instance for lacosamide and brivaracetam (lacosamide ED50 is about 280 mg/day; brivaracetam ED50 is about 50 mg/day).

The term “therapeutically effective amount of BRV” or the term “therapeutically effective amount of LCM” means that at the given dosage of BRV or LCM, there is an anti-convulsive effect in 50% of the population that takes it.

According and Deckers et al. (2000) an isobolographic method used and evaluate interactions among AEDs is considered and be the optimal method for detecting synergy, additivity or antagonism among AEDs in animal models of epilepsy, such as the 6 Hz seizure model in mice. For isobolographic analysis, the experimental (EDmix) and theoretical additive (EDadd) ED50 values are determined from the dose-response curves of combined drugs. ED50 is defined as a dose of a drug protecting 50% of the animals against 6 Hz-induced seizures. ED50mix is an experimentally determined total dose of the mixture of two component drugs, which were administered in the fixed-ratio combination sufficient for a 50% protective effect. Conversely, ED50add represents a total additive dose of two drugs (calculated from the line of additivity), theoretically providing 50% protection against seizures.

The term “interaction index a” refers and the ratio of ED50mix/ED50add. This ratio seems and be a good describer of the strength of interaction between two AEDs in isobolographic analysis (Luszczki et al., 2003; Berenbaum, 1989; Tallarida et al., 1999; Tallarida, 2001, 2002). If ED50mix=ED50add, then α=1. Small derivations of a from 1 may not be considered as significant. If a is smaller than 0.7, this may indicate a synergistic effect. If the index is larger than 1.3, this may indicate an antagonistic effect, and if the index is in between this may indicate purely additive interaction (Luszczki et al., 2003; Kerry et al., 1975; Bourgeois, Wad, 1984, 1988; Bourgeois, 1988).

In a preferred embodiment, the synergistic effect of the pharmaceutical composition of the present invention is defined as a value of the interaction index a of the composition of up and about 0.7, preferably of up and about 0.6, more preferably of up and about 0.5, wherein a >0. Examples for the interaction index a are about 0.1, about 0.2, about 0.3, about 0.4, about 0.5, about 0.6, and about 0.7.

In another preferred embodiment, the synergistic effect of the pharmaceutical composition of the present invention is defined as a value of the benefit index BI of the composition of at least about 1.3, preferably of at least about 1.4, more preferably of at least about 1.5. Examples for the benefit index BI are about 1.3, about 1.4, about 1.5, about 1.6, about 1.7, about 1.8, about 1.9, and about 2.0.

In general, the “fixed-dose ratio of brivaracetam:lacosamide of X:Y, calculated on the ED50 values of the individual compounds brivaracetam and lacosamide” refers to compositions comprising both brivaracetam and lacosamide, wherein the dose of brivaracetam corresponds and X·ED₅₀/(X+Y) of brivaracetam, and the dose of lacosamide corresponds and YED₅₀/(X+Y) of lacosamide, or a multiple of this fixed dose ratio.

Thus, a composition comprising both brivaracetam: lacosamide in a fixed dose ratio of at least X:Y comprises at least X/(at least X+Y) parts of brivaracetam, wherein 1 part is an amount corresponding and the ED50 of brivaracetam, and Y/(at least X+Y) parts of lacosamide, wherein 1 part is an amount corresponding and the ED50 of compound (a), or a multiple of this fixed dose ratio.

The term “multiple of the fixed dose ratio” refers and a composition comprising a larger or a smaller amount of lacosamide and brivaracetam with reference and the amount as defined by the ED50 values, while maintaining the fixed dose ratio. A composition comprising a multiple of the fixed dose ratio as indicated above may thus comprise at least 0.1 times the fixed dose ratio, at least 0.2 times, at least 0.5 times, at least 2 times, at least 5 times, or at least 10 times the fixed dose ratio, or/and at the maximum 100 times the fixed dose ratio, at the maximum 50 times, or at the maximum 20 times the fixed dose ratio.

The weight ratio of brivaracetam and lacosamide is about 1:1.5 or less, preferably about 1:1.45, 1:1.4, 1:1.35, or 1:1.3 or less, more preferably about 1:1.25, 1:1.2, 1:1.15, 1:1.1, 1:1.05, 1:1, 1:0.95, 1:0.9, 1:0.85, 1:0.8, 1:0.75, 1:0.7, 1:0.65, 1:0.6, 1:0.55 or 1:0.5 or less. In certain embodiments, however, dosages wherein the weight ratio of brivaracetam and lacosamide is greater than about 1:1.5 may be preferred, for example, dosages of about 1:1.6, 1:1.7, 1:1.8, 1:1.9, 1:2 or greater. Likewise, in certain embodiments, dosages wherein the ratio of brivaracetam and lacosamide is less than about 1:0.5 may be preferred, for example, about 1:0.45, 1:0.4, 1:0.35, 1:0.3, 1:0.25, 1:0.2, 1:0.15, or 1:0.1 or less.

In yet another preferred embodiment, brivaracetam and lacosamide are present in the pharmaceutical composition of the present invention in a fixed-dose ratio of brivaracetam:lacosamide of about 1:6 and about 6:1, preferably of about 1:3 and about 6:1, more preferably of about 1:1 and about 6:1, even more preferably of about 3:1 and about 6:1, wherein the fixed-dose ratio is calculated on the ED₅₀ values of the individual compounds brivaracetam and lacosamide. Examples for fixed-dose ratios of brivaracetam: lacosamide according and the present invention are fixed-dose ratios of about 1:6, about 1:5, 1:4, about 1:3, about 1:2, and about 1:1. Further examples for fixed-dose ratios according and the present invention are fixed-dose ratios of about 5:1, about 4:1, about 3:1, about 2:1.

It may also be preferred and administer the combined dose (or separate doses simultaneously administered) at the preferred ratio of 1:1 and 1:3 (brivaracetam/lacosamide) or less twice daily, three times daily, four times daily, or more frequently so as and provide the patient with a preferred dosage level per day, for example: 200 mg lacosamide and 200 mg brivaracetam per day provided in two doses, each dose containing 100 mg lacosamide and 100 mg brivaracetam; 150 mg lacosamide and 150 mg brivaracetam per day provided in two doses, each dose containing 75 mg lacosamide and 75 mg brivaracetam; 120 mg lacosamide and 120 mg brivaracetam per day provided in two doses, each dose containing 60 mg lacosamide and 60 mg brivaracetam; 100 mg lacosamide and 100 mg brivaracetam per day provided in two doses, each dose containing 50 mg lacosamide and 50 mg brivaracetam; or 80 mg lacosamide and 80 mg brivaracetam per day provided in two doses, each dose containing 40 mg lacosamide and 40 mg brivaracetam.

The compositions of the present invention are for use as a medicament, in particular for the treatment of an epileptic disorder including epilepsy, epileptogenesis, seizure disorders, convulsions.

Hence, one aspect of the present invention relates and a method of preventing, alleviating and/or treating of an epileptic disorder and/or of epileptic seizures, or of epileptogenesis, and to a method of treating partial onset seizures with and without secondary generalization, or and a method of treating primary generalized tonic clonic seizures, comprising administering and a patient in need thereof an oral fixed dose combination of brivaracetam and lacosamide, wherein said oral fixed dose combination is a tablet (“fixed dose tablet”), and wherein said administration comprises the twice daily administration of one fixed dose tablet per administration, which fixed dose tablet provides the combined release brivaracetam and lacosamide in of the above set out dosages.

In the fixed dosage composition according and the present invention either of both of brivaracetam and lacosamide may be in a particulate state. The term “particulate” refers and a state of matter which is characterized by the presence of discrete particles, pellets, beads or granules irrespective of their size, shape or morphology. When a plurality of particulates is present, these are referred and a multiparticulates. Typically, the particulates have an average size of less than about 3 mm, preferably between about 1 and 3 mm. By “average particle size” it is meant that at least 50% of the particulates have a particle size of less than about the given value, by weight. The particle size may be determined on the basis of the weight average particle size as measured by conventional particle size measuring techniques well known and those skilled in the art. Such techniques include, for example, sedimentation field flow fractionation, photon correlation spectroscopy, light scattering, and disk centrifugation.

The term “small tablets” within the scope of this application denotes tablets with an overall size of less than 15 mm.

The term “minitablets” denotes small tablets with an overall weight of approximately 10 and 50 mg, e.g. approximately 15 and 25 mg, e.g. approximately 18 mg, in their uncoated form. Minitablets are a specific form of multiparticulates. They can be prepared by means known and a person skilled in the art, including preparation from other, smaller multiparticulates, such as granules or beads. The minitablets may have any shape known and the skilled person in the art for tablets, e.g. round e.g. with a diameter of about 1.25 and 3 mm; cyclindrical e.g. having a convex upper face and convex lower face and e.g. with a cylindrical diameter and height independently of each other are from 1 and 3 mm; or biconvex minitablets e.g. whose height to diameter are approximately equal and are from 1.25 and 3 mm.

Preferably, multiparticulates have a controlled release coating. Specifically, if a mixture of multiparticulates brivaracetam and lacosamide are used, the respective multiparticulates comprise different controlled release coatings in order and provide different controlled release profiles.

In one embodiment, brivaracetam and lacosamide may be delivered from a same matrix in a modified or delayed release course i.e. the release of both, brivaracetam and lacosamide, is delayed compared and an IR formulation wherein typically substantially all of the compounds are released after 1 hours, or even after 15 minutes. In case of a modified release (MR) FDC, both brivaracetam and lacosamide may preferably be released from the FDC such that typically no more than about 50 wt %, preferably no more than 45 wt % of each of both compounds is released within one hour, between about 15 wt % and 60 wt % of each of the compounds is released after 2 hours, between about 30 wt % and 85 wt % is released within 4 hours, between about 55 and 100 wt % is released within 8 hours, and/or between 70 and 100 wt % is released within 12 hours, when measured in an in vitro dissolution assay as further specified herein. This may be achieved either by adding modified release polymers or other retarding agents and the matrix or by applying a release modifying coating and an immediate release matrix, or by a combination of release modifying components in the matrix and in the coating. For the purpose of this patent application, the terms “modified release” and “delayed release” are used interchangeably.

Such a modified or delayed release profile may advantageously lead and lower maximum plasma concentrations Cₘₐₓ of the drugs, a reduction of the respective Cₘₐₓ/Cₘᵢₙ ratio, an increased time Tₘₐₓ and reach Cₘₐₓ, and to potentially reduced side effects. Brivaracetam and lacosamide may be released from the matrix with delayed rates such that, for example, the maximum concentration of lacosamide in the patient would be reached at a time Tₘₐₓ which is more than 2 or 3 hours, more than 4 hours, more than 5 hours, or even more than 6 hours after the administration of the FDC and a patient, and/or such that the maximum concentration of brivaracetam may be reached at a time Tₘₐₓ which is more than 3, more than 4, more than 5, or after more than 6 hours after such administration.

In one preferred embodiment, brivaracetam and lacosamide are comprised in the same layer/matrix of the FDC, together with the excipients, and both active ingredients are released from the FDC in modified release mode. The FDC of the present invention may comprise both brivaracetam and lacosamide in a matrix which further comprise at least one agent which delays the release of brivaracetam and lacosamide from said matrix (such agent, a “matrix retarding agent”). The matrix retarding agent(s) may be present in an amount of at least about 1 wt %, at least 1.5 wt %, at least about 2 wt %, at least 3 wt %, at least 4 wt %, at least 5.5 wt %, at least 6 wt %, at least 7 wt %, at least 8 wt %, 9 wt %, at least 10 wt %, at least 12 wt % or at least about 15 wt %, relative and the total weight of the formulation. In order and limit the size of the FDC as much as possible, the matrix retarding agent(s) should be present in the matrix in an amount of less than 50 wt %, preferably less than 45 wt %, or at the most 40 wt %, at the most 35 wt %, or even more preferably at the most 30 wt %, or less, relative and the total weight of the 10 formulation. In particular, the matrix retardation agent(s) may be present in the matrix in an overall amount of between about 10 wt % and 45 wt %, preferably 10 wt % and 40 wt %, more preferably 15 wt % and 35 wt %, even more preferably up and 30 wt % relative and the total weight of the formulation. The matrix retardation agent may be selected from polymeric and non-polymeric matrix retardation agents. Examples for suitable release modifying agents, and suitable drug release profiles can be taken from WO 2012/084126, WO 2012/072556, and from WO 2006/080029. Suitable release modifying agents in the matrix may include hydrophilic polymers (such as e.g. poloxamers, hydroxyethylcellulose, hydroxypropylcellulose (HPC), methylcellulose, carboxymethylcellulose, hydroxyl, propylmethylcellulose (HPMC), polyvinyl pyrrolidone, polyvinyl alcohols, modified starch, pregelatinized starch, hydroxypropyl starch, sodium hyaluronate, alginic acid, alginate salts, carrageenan, chitosan, guar gum, pectin, xanthan gum, and the like), hydrophobic polymers or nonpolymeric substances (such as e.g. C₈-C₃₀ monohydric alcohols, monoglycerides, diglycerides, triglycerides, glycerine esters, hydrogenated castor oil, glyceryl behenate, hydrogenated soybean, oil, lauroyl macrogolglycerides, stearyl macrogolglycerides, glyceryl palmitostearate, cethyl palmitate, glycerol esters of fatty acids and cetyl alcohol and the like), and inert polymers (such as acrylic resins, cellulose derivatives, vinyl acetate derivatives, and non-water soluble polyesters, preferably selected from the group of polyvinyl acetate, ethylcellulose, hydroxypropylmethylcellulose acetate phthalate, hydroxypropylmethylcellulose acetate succinate, shellac, polymethacrylic acid derivatives, methacrylic acid copolymer type A, methacrylic acid copolymer type B, methacrylic acid copolymer type C, ammonio methacrylate copolymer type A (which is a monograph of EUDRAGIT® RL PO is a copolymer of ethyl acrylate, methyl methacrylate and a low content of methacrylic acid ester with quaternary ammonium groups), ammonio methacrylate copolymer type B (which is a monograph of EUDRAGIT® RS 100 which is a copolymer of ethyl acrylate, methyl methacrylate and a low content of methacrylic acid ester with quaternary ammonium groups), neutral ethyl methyl methacrylate copolymer, basic butylated methacrylate copolymer, and the like).

In one preferred aspect, the retardation agent is a hydrophilic matrix retardation agent. Hydrophilic retardation agents have the general advantages of usually becoming completely degraded in the animal body, being well characterized excipients, and showing good technical processability also on larger scale. It has also been shown in the present disclosure that hydrophilic matrix retardation agents are surprisingly well suited and control the dissolution of brivaracetam and lacosamide from the same FDC.

The hydrophilic matrix retardation agent may be selected from the group of gums, cellulose ethers, cellulose esters, and other cellulose derivatives, gelatine, poly-saccharides, starch, starch derivatives, vinyl acetate and its derivatives, vinyl pyrrolidone and its derivatives, and polyethylene glycols. The hydrophilic matrix retardation agents are preferably selected from the group of poloxamers, hydroxyethylcellulose, hydroxypropyl-cellulose (HPC), methylcellulose, carboxymethylcellulose, hydroxypropylmethylcellulose (HPMC), polyvinyl pyrrolidone, polyvinyl alcohols, modified starch, pregelatinized starch, hydroxypropyl starch, sodium hyaluronate, alginic acid, alginate salts, carrageenan, chiandsan, guar gum, pectin, and xanthan gum.

In one preferred aspect, the matrix retardation agent present in the FDC is a hydrophilic polymer material selected from cellulose derivatives such as hydroxyethylcellulose, hydroxypropylcellulose (HPC), methylcellulose, and in particular hydroxypropylmethyl-cellulose (HPMC); such cellulose derivatives having a viscosity of about 50 mPa·s and 200,000 mPa·s in a 2 wt % aqueous solution at 20° C., preferably a viscosity of about 80 mPa·s and about 50,000 mPa·s in a 2 wt % aqueous solution at 20° C. or between about 100 mPa·s and about 25,000 mPa·s, wherein any viscosity referred and in this application is determined by Ubbelohde or Ostwald capillary according and the USP (Edition 24) method <911>. “Viscosity” as used herein is sometimes also termed “apparent viscosity” in the art.

Preferred matrix retarding agents are hydrophilic polymers with a medium viscosity between about 1000 and 25000 mPa·s. Particularly preferred matrix retarding agents are HPMC qualities with a medium viscosity between about 3000 and 25000 mPa·s; such “medium viscosity HPMCs” are commercially available from e.g. Dow Corning under the brand names K4M Premium CR®, E4M Premium CR®, E10M Premium CR® or K15Premium CR®, having viscosities of about 3000 and 6000 mPa·s, about 7500 and 14000 mPa·s, and between about 10000 and 21000 mPa·s, respectively. These medium viscosity HPMCs may be used as sole matrix retardation agents, or may be used in admixture with other hydrophilic polymers having a similar or lower viscosity. If 30 used as sole matrix retarding agent, they may be typically used in amounts of 15 and 30 wt % relative and the total weight of the formulation.

If a high viscosity hydrophilic polymer, in particular a cellulose derivative, e.g. HPC or HPMC, having a viscosity of at least about 30,000 mPa·s, preferably of at least about 50,000 Pa·s or at least about 100,000 mPa·s in 2% aqueous solution is being used as retarding agent, the amount of HPMC in the formulation can surprisingly be as low as about 8 wt % or less, 6 wt % or less, 5 wt % or less, 4 wt % or less, 3 wt % or less or even between 1 wt % and 2 wt % relative and the total weight of the formulation.

In a preferred embodiment, a medium viscosity hydrophilic polymer, preferably HPMC, with a viscosity of about 1000 and 25000 mPa·s, preferably between about 3000 and 25000 mPa·s, and a low viscosity hydrophilic polymer, such as e.g. a HPMC having a viscosity of between about 50 and 1000 mPa·s, or between about 80 and 120 mPa·s can be advantageously used in admixture. A suitable low viscosity HPMC is commercially available from e.g. Dow Corning under the brand name K100LV Premium®. In this embodiment, without wished and be bound and any theory, the low viscosity polymer, e.g. the HPMC, is thought and modulate or fine tune the stronger retarding effect of the medium viscosity hydrophilic polymer.

Pharmaceutical compositions of the invention may optionally comprise one or more pharmaceutically acceptable binding agents or adhesives as excipients, particularly for tablet formulations. Such binding agents and adhesives preferably impart sufficient cohesion and the powder being tableted and allow for normal processing operations such as sizing, lubrication, compression and packaging, but still allow the tablet and disintegrate and the composition and be absorbed upon ingestion. Such binding agents may also prevent or inhibit crystallization or recrystallization of a APIs of the present invention once the salt has been dissolved in a solution. Suitable binding agents and adhesives include, but are not limited and, either individually or in combination, acacia; tragacanth; sucrose; gelatin; glucose; starches such as, but not limited and pregelatinized starches (e.g. National TM 1511 or National Tm1500); celluloses such as, but not limited and, methylcellulose and carmellose sodium (e.g. Tylose), alginic acid and salts of alginic acid; magnesium aluminum silicate; PEG; guar gum; polysaccharide acids; benandnites; povidone, for example povidone K-15,K-30 and K-29/32; polymethacrylates; HPMC; hydroxypropyl-cellulose (e.g. Klucel of Aqualon); and ethylcellulose (e.g. Ethocel™ of the Dow Chemical Company). Such binding agents and/or adhesives, if present, constitute in total about 0.5% and about 25%, preferably about 0.75% and about 15%, and more preferably about 1% and about 10%, of the total weight of the pharmaceutical composition.

Many of the binding agents are polymers comprising amide, ester, ether, alcohol or ketone groups and, as such, are preferably included in pharmaceutical compositions of the present invention. Polyvinylpyrrolidones such as povidone K-30 are especially preferred. Polymeric binding agents can have varying molecular weight, degrees of crosslinking, and grades of polymer. Polymeric binding agents can also be copolymers, such as block co-polymers that contain mixtures of ethylene oxide and propylene oxide units. Variation in these units ratios in a given polymer affects properties and performance. Examples of block co-polymers with varying compositions of block units are Poloxamer 188 and Poloxamer 237 (BASF Corporation).

Pharmaceutical compositions of the invention optionally comprise one or more pharmaceutically acceptable wetting agents as excipients. Such wetting agents are preferably selected and maintain the APIs in close association with water, a condition that is believed and improve bioavailability of the composition. Such wetting agents can also be useful in solubilizing or increasing the solubility of the APIs, i.e. of lacosamide and brivaracetam.

Non-limiting examples of surfactants that can be used as wetting agents in pharmaceutical compositions of the invention include quaternary ammonium compounds, for example benzalkonium chloride, benzethonium chloride and cetylpyridinium chloride, dioctylsodium sulfosuccinate, polyoxyethylene alkylphenyl ethers, for example nonoxynol, nonoxynol, and degrees Candxynol, poloxamers (polyoxyethylene and polyoxypropylene blockcopolymers polyoxyethylene fatty acid glycerides and oils, for example polyoxyethylene (caprylic/capric mono- and diglycerides (e.g., Labrasol of Gattefosse), polyoxyethylene castor oil and polyoxyethylene, hydrogenated castor oil; polyoxyethylene alkyl ethers, for example polyoxyethylene cetostearyl ether, polyoxyethylene fatty acid esters, for example polyoxyethylene stearate, polyoxyethylene sorbitan esters, for example polysorbate and polysorbate, propylene glycol fatty acid esters, for example propylene glycol laurate (e.g. Lauroglycol of Gattefosse), sodium lauryl sulfate, fatty acids and salts thereof, for example oleic acid, sodium oleate or triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof. Such wetting agents, if present, constitute in total about 0.25% and about 15%, preferably about 0.4% and about 10%, and more preferably about 0.5% and about 5%, of the total weight of the pharmaceutical composition.

Wetting agents that are anionic surfactants are preferred. Sodium lauryl sulfate is a particularly preferred wetting agent. Sodium lauryl sulfate, if present, constitutes about 0.25% and about 7%, more preferably about 0.4% and about 4%, and still more preferably about 0.5% and about 2%, of the total weight of the pharmaceutical composition.

Pharmaceutical compositions of the invention optionally comprise one or more pharmaceutically acceptable lubricants (including anti-adherents and/or glidants) as excipients. Suitable lubricants include, but are not limited and, either individually or in combination, glycerylbehapate (e.g., Compritol TM 888 of Gattefosse); stearic acid and salts thereof, including magnesium, calcium and sodium stearate; hydrogenated vegetable oils (e.g. Sterotexof Abitec); colloidal silica; talc; waxes; boric acid; sodium benzoate; sodium acetate; sodium fumarate; sodium chloride; DL-leucine; PEG (e.g. Carbowax 4000 and Carbowax 6000 of the Dow Chemical Company); sodium oleate; sodium lauryl sulfate; and magnesium lauryl sulfate. Such lubricants, if present, constitute in total about 0.1% and about 10%, preferably about 0.2% and about 8%, and more preferably about 0.25% and about 5%, of the total weight of the pharmaceutical composition.

Magnesium stearate is a preferred lubricant used, for example, and reduce friction between the equipment and granulated mixture during compression of tablet formulations.

Suitable anti-adherents include, but are not limited and, talc, cornstarch, DL-leucine, sodium lauryl sulfate and metallic stearate. Talc is a preferred anti-adherent or glidant used, for example, and reduce formulation sticking and equipment surfaces and also and reduce static in the blend. Talc, if present, constitutes about 0.1% and about 10%, more preferably about 0.25% and about 5%, and still more preferably about 0.5% and about 2%, of the total weight of the pharmaceutical composition.

Glidants can be used and promote powder flow of a solid formulation. Suitable glidants include, but are not limited and, colloidal silicon dioxide, starch, talc, tribasic calcium phosphate, powdered cellulose and magnesium trisilicate. Colloidal silicon dioxide is particularly preferred.

Other excipients such as colorants, flavors and sweeteners are known in the pharmaceutical art and can be used in pharmaceutical compositions of the present invention. Tablets can be coated, for example with an enteric coating, or uncoated.

Compositions of the invention can further comprise, for example, buffering agents.

Optionally, one or more effervescent agents can be used as disintegrants and/or and enhance organoleptic properties of pharmaceutical compositions of the invention. When present in pharmaceutical compositions of the invention and promote dosage form disintegration, one or more effervescent agents are preferably present in a total amount of about 30% and about 75%, and preferably about 45% and about 70%, for example about 60%, by weight of the pharmaceutical composition.

According and a particularly preferred embodiment of the invention, an effervescent agent, present in a solid dosage form in an amount less than that effective and promote disintegration of the dosage form, provides improved dispersion of the API in an aqueous medium. Without being bound by theory, it is believed that the effervescent agent is effective and accelerate dispersion of the API from the dosage form in the gastrointestinal tract thereby further enhancing absorption and rapid onset of therapeutic effect. An effervescent agent is preferably present in the composition in an amount of about 1% and about 20%, more preferably about 2.5% and about 15%, and still more preferably about 5% and about 10%, by weight of the pharmaceutical composition.

An “effervescent agent” herein is an agent comprising one or more compounds which, acting together or individually, evolve a gas on contact with water. The gas evolved is generally oxygen or, most commonly, carbon dioxide. Preferred effervescent agents comprise an acid and a base that react in the presence of water and generate carbon dioxide gas. Preferably, the base comprises an alkali metal or alkaline earth metal carbonate or bicarbonate and the acid comprises an aliphatic carboxylic acid.

Non-limiting examples of suitable bases as components of effervescent agents useful in the invention include carbonate salts (e.g. calcium carbonate), bicarbonate salts (e.g., sodium bicarbonate), sesquicarbonate salts, and mixtures thereof. Calcium carbonate is a preferred base.

Non-limiting examples of suitable acids as components of effervescent agents and/or solid organic acids useful in the invention include citric acid, tartaric acid (as D-, L-, or D/L-tartaric acid), malic acid (as D-, L-, or DL-malic acid), maleic acid, fumaric acid, adipic acid, succinic acid, acid anhydrides of such acids, acid salts of such acids, and mixtures thereof. Citric acid is a preferred acid.

In a preferred embodiment of the invention, where the effervescent agent comprises an acid and a base, the weight ratio of the acid and the base is about 1:100 and about 100:1, more preferably about 1:50 and about 50:1, and still more preferably about 1:10 and about 10:1. In a further preferred embodiment of the invention, where the effervescent agent comprises an acid and a base, the ratio of the acid and the base is approximately stoichiometric.

Excipients which solubilize APIs typically have both hydrophilic and hydrophobic regions, or are preferably amphiphilic or have amphiphilic regions. One type of amphiphilic or partially-amphiphilic excipient comprises an amphiphilic polymer or is amphiphilic polymer. A specific amphiphilic polymer is a polyalkylene glycol, which is commonly comprised ethylene glycol and/or propylene glycol subunits. Such polyalkylene glycols can be esterified at their termini by a carboxylic acid, ester, acidanhyride or other suitable moiety. Examples of such excipients include poloxamers (symmetric block copolymers of ethylene glycol and propylene glycol; e.g., poloxamer 237), polyalkyene glycolated esters of tocopherol (including esters formed from a di- or multi-functional carboxylic acid; e.g., d-alpha-andcopherol polyethylene glycol-1000 succinate), and macrogolglycerides (formed by alcoholysis of an oil and esterification of a polyalkylene glycol and produce a mixture of mono-, di- and tri-glycerides and mono- and di-esters; e.g. stearoyl macrogol-32 glycerides). Such pharmaceutical compositions are advantageously administered orally.

Pharmaceutical compositions of the present invention can comprise about 10% and about 50%, about 25% and about 50%, about 30% and about 45%, or about 30% and about 35% by weight of a API; about 10% and about 50%, about 25% and about 50%, about 30% and about 45%, or about 30% and about 35% by weight of an excipient which inhibits crystallization in aqueous solution, in simulated gastric fluid, or in simulated intestinal fluid and about 5% and about 50%, about 10% and about 40%, about 15% and about 35%, or about 30% and about 35% by weight of a binding agent.

Solid dosage forms of the invention can be prepared by any suitable process, not limited and processes described herein.

An illustrative process comprises (a) a step of blending an API of the invention with one or more excipients and form a blend, and (b) a step of tableting or encapsulating the blend and form tablets or capsules, respectively.

In a preferred process, solid dosage forms are prepared by a process comprising (a) a step of blending a API of the invention with one or more excipients and form a blend, (b) a step of granulating the blend and form a granulate, and (c) a step of tableting or encapsulating the blend and form tablets or capsules respectively. Step (b) can be accomplished by any dry or wet granulation technique known in the art, but is preferably a dry granulation step. A salt of the present invention is advantageously granulated and form particles of about 1 micrometer and about 100 micrometer, about 5 micrometer and about 50 micrometer, or about 10 micrometer and about 25 micrometer. One or more diluents, one or more disintegrants and one or more binding agents are preferably added, for example in the blending step, a wetting agent can optionally be added, for example in the granulating step, and one or more disintegrants are preferably added after granulating but before tableting or encapsulating. A lubricant is preferably added before tableting. Blending and granulating can be performed independently under low or high shear. A process is preferably selected that forms a granulate that is uniform in API content, that readily disintegrates, that flows with sufficient ease so that weight variation can be reliably controlled during capsule filling or tableting, and that is dense enough in bulk so that a batch can be processed in the selected equipment and individual doses fit into the specified capsules or tablet dies.

In an alternative embodiment, solid dosage forms are prepared by a process that includes a spray drying step, wherein an API is suspended with one or more excipients in one or more sprayable liquids, preferably a non-protic (e.g., non-aqueous or non-alcoholic) sprayable liquid, and then is rapidly spray dried over a current of warm air.

A granulate or spray dried powder resulting from any of the above illustrative processes can be compressed or molded and prepare tablets or encapsulated and prepare capsules.

Conventional tableting and encapsulation techniques known in the art can be employed. Where coated tablets are desired, conventional coating techniques are suitable.

Excipients for tablet compositions of the invention are preferably selected and provide a disintegration time of less than about 30 minutes, preferably about 25 minutes or less, more preferably about 20 minutes or less, and still more preferably about 15 minutes or less, in a standard disintegration assay.

### EXAMPLES

### Example 1: 6 Hz Seizure Test

The aim of this study was and investigate potential interactions between Lacosamide and Brivaracetam in the 6 Hz seizure model in mice using the isobolographic analysis. According and Deckers et al. (2000) an isobolographic method is used to evaluate interactions among AEDs and it is considered to be the optimal method for detecting synergy, additivity or antagonism among AEDs in animal models of epilepsy. The 6 Hz seizure model was performed as previously described (Kaminski et al., 2004). Briefly, mice were stimulated through corneal electrodes connected to an electrical stimulator (ECT Unit 5780, Ugo-Basile, Comerio, Italy) delivering a constant current (0.2 ms duration monopolar rectangular pulses at 6 Hz for 3 s). A drop of saline containing 0.4% oxybuprocaine hydrochloride (Unicaine, Thea, France) was applied on the eyes before stimulation and provide local anesthesia and ensure optimal current conductivity. During the stimulation, each mouse was manually restrained then gently released into the observation cage (38×26×14 cm) immediately after the current application. The seizures were often preceded by a brief period (˜2-3 s) of locomotor agitation (running and jumping). The animals then exhibited immobility associated with rearing, automatisms, forelimb clonus, twitching of the vibrissae and sometimes Straub tail. The animals (10 mice per group) were observed for 30 s following the electrical stimulation. The main seizure endpoint was the duration of the immobility. Mice resuming normal behavior within 7 s after the end of the stimulation were considered as not displaying seizure behavior.

Animals

The experiments were performed on adult male NMRI mice (Charles River, France) weighing between 24 and 36 g. The mice were kept in colony cages with free access and food and water, under standard laboratory conditions with natural light-dark cycle. After 1 week adaptation the animals were randomly assigned and experimental groups consisting of ten mice. Each mouse was used only once. All experiments were performed between 9 am and 4 pm. Procedures involving animals and their care were conducted in accordance with current European Community regulations.

Drugs

Lacosamide and Brivaracetam from UCB Pharma Sprl were dissolved in 0.5% methylcellulose and administered intraperitoneally (i. p.) in a volume of 0.2 ml/20 g body weight (LCM, BRV—30 min before the test).

Fresh drug solutions were prepared ex tempore on each day of experimentation. These pretreatment times before testing of BRV/LCM were based on information about their biologic activity from the literature.

Data Analysis

The isobolographic analysis is based on a comparison of equieffective drug doses. In the present study, interactions between drugs, as regards their anticonvulsant efficacy against 6 Hz seizure test were evaluated isobolographically according and the procedure elaborated by Tallarida (1992); Porreca et al. (1990); Luszczki et al. (2006). The experimental (ED50mix) and theoretical additive (ED50add) were determined from the dose-response curves of combined drugs (Tallarida et al., 1997). ED50 is defined as a dose of a drug protecting 50% of the animals against 6 Hz-induced seizures. ED50mix is an experimentally determined dose of the mixture of two component drugs, which were administered in the fixed-ratio combination sufficient for a 50% protective effect. Conversely, ED50add represents an additive dose of two drugs (calculated from the line of additivity), theoretically providing 50% protection against seizures. The respective 95% confidence limits of ED50mix were calculated according and Litchfield and Wilcoxon (1949), and these of ED50add according and Tallarida and Murray (1987), and subsequently transformed to the standard error of mean (SEM), according to the procedure described in detail by Luszczki, et al. (2003).

To estimate the types of interactions, three fixed-dose ratios of the drugs were examined as follows 1:3, 1:1, and 3:1 in the 6 Hz-induced seizures. To visualize the types of interactions between Lacosamide and Brivaracetam, the isoboles were drawn by plotting the points reflecting the respective doses of LCM (on Y-axis) and doses of BRV on the X-axis. The straight line connecting ED50 values for the two tested drugs administered alone against 6 Hz-induced seizures, represents the theoretic isobole for additivity. If experimentally determined data points, reflecting the combinations of various fixed ratios, lie on this line the drug effects are additive (no interaction). If the points fall significantly below the additive line, the two component drugs act synergistically. Conversely, antagonism may be recognized if these points are localized above the additive isobole.

Moreover, an interaction index for various fixed-ratio combinations of Lacosamide and Brivaracetam in the 6 Hz-test was calculated as a ratio ED50mix/ED50add. This ratio seems and be a good describer of the strength of interaction between Lacosamide and Brivaracetam in isobolographic analysis (Luszczki et al., 2003; Berenbaum, 1989; Tallarida et al., 1999; Tallarida, 2001, 2002). If the index is smaller than 0.7, this indicates a synergistic effect. If the index is larger than 1.3, this indicate an antagonistic effect, and if the index is in between this indicates purely additive interaction (Luszczki et al., 2003; Kerry et al., 1975; Bourgeois, Wad, 1984, 1988; Bourgeois, 1988). However, since this is an arbitrary estimate, a more objective way is to perform a statistical comparison. This was done by using ED50add vs. ED50mix values and comparing them with Student's t-test as previously described (Luszczki et al., 2003; 2004).

Results: AED anticonvulsant effects against 6 Hz-induced seizures in mice.

Lacosamide and Brivaracetam produced dose-dependent anticonvulsant effects against 6 Hz seizure in mice. The ED50 values for the drugs administered alone are presented in Table 1.

TABLE 1

| Drug | ED_{50add} ± SEM (mg/kg) |
|---|---|
| Brivaracetam | 14.2 ± 1.1 |
| Lacosamide | 8.8 ± 1.1 |

Isobolographic analysis of interactions between Lacosamide and Brivaracetam in the 6 Hz-seizure model.

Based on ED50 values determined for Lacosamide and Brivaracetam individually, a theoretical additive ED50 for drug mixtures (ED50add values) was calculated for three fixed-ratios (1:3, 1:1 and 3:1). Subsequently, the experimental ED50mix values were determined for the same fixed-ratio combinations in the 6 Hz seizure test (Table 2). The isobolographic analysis demonstrated synergistic interactions were noted for all doses of BRV, combined with LCM.

TABLE 2

| Fixed ratio | ED_{50add} ± SEM | ED₅₀ₘᵢₓ ± SEM | Interaction | P | |
|---|---|---|---|---|---|
| Brivaracetam:Lacosamide | (mg/kg) | (mg/kg) | index (α) | value | Interpretation |
| 1:3 | 10.1 ± 1.1 | 3.2 ± 0.8 | 0.32 | <0.01 | Synergism |
| 1:1 | 11.5 ± 1.1 | 4.2 ± 0.8 | 0.36 | <0.01 | Synergism |
| 3:1 | 12.8 ± 1.1 | 6.9 ± 1.1 | 0.53 | <0.05 | Synergism |

Discussion:

This study demonstrates that LCM fully protected mice against 6 Hz seizures with an ED50 of 8.8 mg/kg. This dose corresponds well with the ED50 (9.9 mg/kg) determined in the anticonvulsant drug screening program of NINDS but is 2-3 times higher than the ED50 needed for protection of maximal electroshock seizures in mice and rats (Rogawski et al., 2015).

The 6 Hz test is regarded a model for treatment resistant seizures e.g. due the fact that many AEDs do not provide adequate protection against these seizures (Barton et al., 2001). Our data confirm the differences in the pharmacological profile of the MES and 6 Hz seizure models. Barton et al. (2001) used the immediate early gene c-Fos as a marker of seizure induced neuronal activation and showed that 6 Hz induced seizures result in a clearly different pattern of neuronal activation than that observed following maximal electroshock or PTZ induced seizures. Duncan and Kohn (2004) showed by using the 2-deoxy glucose technique that this specific pattern of neuronal activation was attenuated by lacosamide while the drug had no effect on basal patterns.

The isobolographic analysis revealed that LCM acts synergistically with BRV across all examined fixed ratios.

The Lacosamide and Brivaracetam drug combinations studied exhibited no infra-additive effects (antagonism between drugs for anti-seizure efficacy) or potentiation of toxicities. In no cases in which there was potentiation of anti-seizure activities there was also potentiation of acute neurotoxicity. This is, of course, a desirable interaction for any drug combination since the result may be an improved margin of safety.

We can suggest some mechanism underlying the different types of interactions observed between Lacosamide and Brivaracetam. First of all, one can exclude pharmacokinetic effects as the reason for the additive or synergistic effects although plasma levels of BRV/LCM have not been determined. LCM does not inhibit or induce a large variety of drug metabolizing enzymes, nor is it metabolized and a significant extent by one of them. Additionally, clinical population pharmacokinetic analysis provided no evidence for any effect of LCM on plasma levels of BRV or vice versa. Thus the interactions found in the present study are purely of pharmacodynamic nature.

The mechanisms of action underlying the nature of the synergistic or additive interaction between Lacosamide and Brivaracetam are unknown. According and Deckers et al. (2000), synergistic interactions are likely between drugs with different mechanisms of action, and additivity may be expected for drugs sharing similar mechanisms.

### Example 2: Amygdala Kindling in Rats

Electrical kindling of brain regions (amygdala, hippocampus) is a widely used model of TLE. Kindling is the progressive increase of brain excitability upon repeated administration of an initially sub-convulsive stimulus, which leads and the occurrence of a permanent epileptic focus in the stimulated brain area (McIntyre et al., 2002). Fully kindled rats are characterized by the induction of complex partial and secondarily generalized seizures upon brief electrical stimulation.

Male Sprague-Dawley rats (Charles River, France) weighing 270-370 g at the initiation of surgery were used. They were anesthetized with intramuscular (i.m.) injections with Domitor (medetomidine 0.5 mg/kg)/Imalgene (ketamine 50 mg/kg). An i.m. injection of an antibiotic, Extencillin (0.5 ml), was also performed, followed by a local injection of Carprofen under the skin of the skull. The rats were then implanted with a bipolar stimulation/recording electrode in the right basolateral amygdala with the following coordinates measured from bregma: AP-2.3 mm, L-4.8 mm, V-8.5 mm (Paxinos and Watson, 1982). The electrode consisted of two twisted Teflon-coated stainless steel wires. An electrode in the left occipital cortex served as the indifferent reference electrode. Bipolar, reference and ground electrodes were connected and plugs and the assembly and anchor screws were held in place with dental acrylic cement applied and the exposed skull surface. Antisedan (atipamezole) was administered i.m. to facilitate awakening on a heating pad.

After a postoperative period of three weeks, the rats were stimulated once daily, five days per week, in the amygdala with 500 μA-1 ms monophasic square wave pulses, 50 Hz for 1 s (Löscher et al., 1986). Kindling was defined as the occurrence of at least ten consecutive stage 4 or 5 seizures according to Racine's scale (Racine, 1972). Fully kindled rats were stimulated once a week in order and ensure persistence of the kindled state. Each behavioral seizure score and duration of afterdischarge was noted for every animal. An afterdischarge is defined as an EEG activity having an amplitude of at least twice the amplitude of the pre-stimulus recording and a frequency greater than 1 Hz.

Drugs:

Brivaracetam was synthesized by UCB Biopharma Sprl and dissolved in water and final concentrations of 3, 8 and 12 mg/ml and give a solution of pH 5.

Lacosamide was synthesized by UCB Biopharma Sprl and suspended in Tween 80 0.1% in water and final concentrations of 3, 8 and 12 mg/ml and give suspensions of pH 4.5.

Testing:

Different groups of rats (n=8) were stimulated on day 1 in order and assess individual behavioral seizure score and the duration of the afterdischarge.

Dose response curves of Lacosamide and Brivaracetam alone were performed on day 3, 1 h after oral administration (5 ml/kg) of increasing doses of compounds. The proportion of animals protected against generalized seizures (scores 3-5) was noted for each dose.

Interaction studies were performed by combining ineffective doses of Lacosamide and Brivaracetam. One week washout period was allowed between two different treatment paradigms in the same groups of rats and avoid alterations in potency due drug accumulation.

Results:

BRV alone (20 or 40 mg/kg) did not produce any significant effect on the protection of animals against secondarily generalized seizures or seizure score (Table 3).

LCM (20, 40 and 60 mg/kg) also did not have any significant effect on these parameters of the amygdala kindling model (Table 3). However, combinations with BRV (20 mg/kg) and LCM (20 mg/kg), which correspond to 1:1 ratio based on administered doses in mg/kg produced significant effect on the reduction of seizure severity, but not protection against secondarily generalized seizures. At higher doses, 40 mg/kg of both Lacosamide and Brivaracetam (1:1 ratio based on administered doses in mg/kg) a significant effect on both seizure severity and protection against secondarily generalized seizures was observed. Similarly, combinations with BRV (20 mg/kg) and LCM (60 mg/kg), which correspond and 1:3 ratio based on administered doses in mg/kg, produced significant effect on both seizure severity and protection against secondarily generalized seizures (Table 3).

TABLE 3

| | Protection | Seizure score |
|---|---|---|
| | n/N | Mean ± SEM |
| BRV 20 mg/kg | 1/8 | 4.25 ± 0.41 |
| LCM 20 mg/kg | 0/8 | 4.75 ± 0.16 |
| COMBO | 3/8 | 2.75 ± 0.37*** |
| BRV 40 mg/kg | 1/8 | 4.63 ± 0.38 |
| LCM 40 mg/kg | 1/8 | 4.38 ± 0.50 |
| COMBO | 6/8^{#} | 1.75 ± 0.31*** |
| BRV 20 mg/kg | 1/8 | 4.25 ± 0.41 |
| LCM 60 mg/kg | 0/8 | 4.38 ± 0.26 |
| COMBO | 4/8^{#} | 2.13 ± 0.55** |

^{#}p < 0.05 Fisher's exact probability test versus LCM alone
**p < 0.01,
***p < 0.001 unpaired t-test versus LCM alone

Discussion:

The results obtained in the amygdala kindling experiment indicate that fixed dose combinations of Lacosamide and Brivaracetam at doses that do not exert any significant anticonvulsant effect, when administered alone, lead to a dramatic increase in efficacy against seizures. As such these results clearly illustrate that the synergistic effects between Lacosamide and Brivaracetam on potency, which were observed in the 6 Hz model (Example 1), translate into increased efficacy, i.e. stronger anti-convulsant effect, when administered at fixed dose combinations.

### REFERENCES

Kaminski R M, Livinghood M R, Rogawski M A. Allopregnanolone analogs that positively modulate GABAA receptors protect against partial seizures induced by 6-Hz electrical stimulation in mice. Epilepsia 2004; 45:864-867.
Stöhr T, Kupferberg H J, Stables J P, Choi D, Harris R H, Kohn H, Walton N, White H S. Lacosamide, a novel anti-convulsant drug, shows efficacy with a wide safety margin in rodent models for epilepsy. Epilepsy Res. 2007 May; 74(2-3):147-54.
Rogawski M A, Tofighy A, White H S, Matagne A, Wolff C. Current understanding of the mechanism of action of the antiepileptic drug lacosamide. Epilepsy Res. 2015 February; 110C:189-205.
Racine R J. Modification of seizure activity by electrical stimulation. II. Motor seizure. Electroencephalogr. Clin. Neurophysiol., 1972, 32:295-99.
Paxinos G. and Watson C. The Rat Brain in Stereotaxic Coordinates. Academic Press Australia, 1982.
Loscher W., Jäckel R., Czuczwar S J. Is amygdala kindling in rat a model for drug-resistant partial epilepsy? Exp. Neurol., 1986, 93:211-26.
McIntyre D C., Poulter M O., Gilby K. Kindling: some old and some new. Epilepsy Res., 50, 79-82.
Matagne A, Margineanu D G, Kenda B, Michel P, Klitgaard H. Anti-convulsive and anti-epileptic properties of brivaracetam (ucb 34714), a high-affinity ligand for the synaptic vesicle protein, SV2A. Br J Pharmacol. 2008 August; 154(8):1662-71.

1. A pharmaceutical oral fixed-dose combination comprising;
a) 5-150 mg brivaracetam
b) 5-250 mg lacosamide, and
c) a pharmaceutically acceptable excipient.

2. A pharmaceutical oral fixed-dose combination according to claim 1, comprising
a) 25-100 mg brivaracetam
b) 100-200 mg lacosamide, and
c) a pharmaceutically acceptable excipient.

3. A pharmaceutical oral fixed-dose combination according to claim 1, comprising
a) 50-80 mg brivaracetam
b) 120-150 mg lacosamide, and
c) a pharmaceutically acceptable excipient.

4. The fixed dose combination according to claim 1, wherein the fixed dose combination is in tablet form.

5. The fixed dose combination according to claim 1, wherein the ratio brivaracetam:lacosamide is from about 1:6 and to about 6:1 on a weight to weight basis.

6. The fixed dose combination according to claim 5, wherein the ratio of brivaracetam:lacosamide of is about 1:1, 1:2, or 1:3 on a weight to weight basis.

7. The fixed dose combination according to claim 1, the fixed dose combination contains pharmaceutically acceptable excipients that provide an extended release of at least one of lacosamide or brivaracetam.

8. A method of treating epileptic disorder in a subject in need thereof, the method comprising administering an effective amount of the fixed dose combination according to claim 1 to the subject.

9. The method according to claim 9, wherein a daily dose of less than 250 mg of lacosamide and less than 100 mg brivaracetam is administered.

10. A method of preventing, alleviating, or treating an epileptic disorder in a subject in need thereof, the method comprising the fixed dose combination according to claim 1 once or twice daily, and wherein the daily dose administered is between 20 to 150 mg brivaracetam and between 50 to 250 mg lacosamide.

11. The method according to claim 10 wherein the daily dose of lacosamide administered is between 120 to 200 mg.

12. The method according to claim 10, wherein the daily dose of brivaracetam administered is between 30 to 80 mg.
